(19) United States
(12) Patent Application Publication
(10) Pub. No.: US 2023/0374615 A1
(43) Pub. Date: Nov. 23, 2023

(54) COMPOSITIONS, KITS, METHODS FOR DETECTING AND IDENTIFYING PATHOGENS THAT CAUSE RESPIRATORY TRACT INFECTIONS AND USE THEREOF

(71) Applicant: SANSURE BIOTECH INC., Changsha (CN)

(72) Inventors: Lizhong DAI, Changsha (CN); Xiaoliang CHEN, Changsha (CN); Xiaomei REN, Changsha (CN); Zhongping DENG, Changsha (CN); Jia LIU, Changsha (CN)

(21) Appl. No.: 18/332,805

(22) Filed: Jun. 12, 2023

Related U.S. Application Data
(63) Continuation of application No. PCT/CN2022/117972, filed on Sep. 9, 2022.

(30) Foreign Application Priority Data
Nov. 16, 2021 (CN) 202111356118.4

(51) Int. Cl. C12Q 1/70

(52) U.S. Cl. CPC C12Q 1/701; C12Q 2600/16

(57) ABSTRACT

The disclosure relates to the field of molecular biological detection, specifically, to the field of detection of pathogens that cause respiratory tract infections. More specifically, the disclosure is capable of simultaneously detecting Influenza A virus, Influenza B virus, Rhinovirus, Respiratory adenovirus, Respiratory syncytial virus and Mycoplasma pneumoniae. Using the compositions of the disclosure, it is possible to detect six pathogens that cause respiratory tract infections at the same time and to identify specifically which pathogen(s) causes the infections. Also, it has a higher detectable rate than that of the existing composition, and is more accurate in detection.

### CROSS-REFERENCE TO RELATED APPLICATIONS

This disclosure is a continuation of International application No. PCT/CN2022/117972, filed on Sep. 9 2022, which claims priority to Chinese patent application CN202111356118.4 filed on Nov. 16, 2021. All of the aforementioned patent applications are hereby incorporated by reference in their entireties.

### REFERENCE TO AN ELECTRONIC SEQUENCE LISTING

The contents of the electronic sequence listing (CU750SequenceListing.xml; Size: 29,796 bytes; and Date of Creation: Jun. 9, 2023) is herein incorporated by reference in its entirety.

### TECHNICAL FIELD

The disclosure relates to the field of molecular biological detection, in particular, to the field of detection of pathogens that cause respiratory tract infections. More specifically, the disclosure is capable of simultaneously detecting Influenza A virus, Influenza B virus, Rhinovirus, Respiratory adenovirus, and Respiratory syncytial virus and Mycoplasma pneumoniae.

### BACKGROUND

Infectious respiratory system diseases are the most common and frequently-occurring diseases in clinical practice. These diseases have similar clinical symptoms and epidemiological characteristics, and thus is difficult to be identified and determined for the pathogen species infected according to clinical symptoms and routine laboratory tests. Respiratory pathogens can be transmitted through the air. The pathogens that cause acute respiratory diseases have the characteristics of strong infectivity, rapid spread, short incubation period, and acute onset, and may cause a wide range of acute diseases of upper and lower respiratory tract, which seriously endanger human health. Common respiratory pathogens include the followings.

Influenza virus, also referred to as flu virus, is an RNA virus that causes influenza in humans and animals. Human influenza viruses, classified into three types of A, B, and C, are the pathogens of influenza (flu). Avian influenza (AI), also known as bird flu, is an infectious disease caused by a subtype of Influenza A virus (also known as avian influenza virus). Taxonomically, the influenza virus belongs to the Influenza virus genus in the family Orthomyxoviridae. It can cause acute upper respiratory tract infection and spread rapidly through the air, and often leads to periodic pandemics that occur globally, such as the “Spanish Influenza” that killed more than 20 million people worldwide in 1918-1919, the “Asian Influenza” that occurred in 1957, and the “Hong Kong Influenza” that occurred in 1968 and the “Russian Influenza” occurred in 1977, as well as the H7N9 avian influenza occurred in 2013.

Respiratory syncytial virus (RSV) is an RNA virus belonging to the Pneumonia genus in the family Paramyxoviridae. Clinical studies have shown that RSV is the most common pathogen causing viral pneumonia. RSV infection is more common in infants under three years of age, with the main symptoms being high fever, rhinitis, pharyngitis and laryngitis, and later manifesting as bronchiolitis and pneumonia, and may be complicated by otitis media, pleurisy, myocarditis and so on in a small number of sick children. Infection in adults and older children mainly manifests upper respiratory tract infections.

Adenovirus (Adv) belongs to the family Adenoviridae. According to their different immunological, biological and biochemical characteristics, adenovirus is classified into 7 subspecies from A to G, with a total of 52 serotypes. Different serotypes have different organ affinities and cause corresponding clinical manifestations, and may infect the respiratory tract, gastrointestinal tract, urethra, bladder, eyes, liver, and the like.

Human Rhinovirus (HRV) is a non-enveloped, single-stranded RNA virus belonging to the genus Enterovirus in the Picornaviridae family. Human Rhinovirus has the same morphological structure and genome structure as enterovirus. It forms a spherical shape with a diameter of 15-30 nm, and has a nucleocapsid with icosahedral symmetry and no envelope. The genome is single-stranded, positive-strand RNA that is about 7500 nucleotides in length, and includes only one reading frame that is about 6500 nucleotides in length; the reading frame is initiated 611 nucleotides from 5′ end, and stops 42 nucleotides from the poly A tail. Human Rhinovirus is a highly contagious and ubiquitous virus that is usually transmitted through direct contact with respiratory droplets/microdroplets, and also through contaminated surfaces, including person-to-person direct contact. HRV has three distinct subgroups A, B, and C consisting of 80, 30, and 56 types, respectively. It is the most serotyped virus among human infectious viruses. HRV's genetic diversity (>150 types) makes it very difficult to develop an effective vaccine. A certain degree of immune protection may be gained after infection, however, previous infection cannot provide complete immunity due to too many serotypes of HRV, that is to say, other serotyped virus may still lead to reinfection. HRV is the leading cause of the “common cold” manifesting as rhinorrhea, sore throat, cough, and malaise. HRV also causes many other respiratory diseases, such as asthma, chronic obstructive pulmonary disease, otitis media, angina, pneumonia and acute bronchiolitis. HRV co-infection with other respiratory viruses, more commonly Respiratory syncytial virus, adenovirus, and the like, occurs frequently and aggravate the patient's condition, seriously affecting the life quality of the patients.

Mycoplasma pneumonia (MP) is a common pathogen of community-acquired pneumonia. Infection with MP can cause atypical pneumonias with pathological changes mainly manifesting as interstitial pneumonia, sometimes complicated by bronchial pneumonia. MP is mainly transmitted through droplets, has an incubation period of 2 to 3 weeks and presents the highest incidence in adolescents. MP causes mild clinical symptoms with only general respiratory symptoms such as headache, sore throat, fever and cough in most patients, and a few would have a persistent high fever, severe cough, and a rapidly progressing disease, leading to severe or critical conditions such as respiratory failure, multiple organ dysfunction in a short period of time.

Conventionally, some technologies are used for detecting respiratory tract infection pathogens, such as Chinese invention patent CN113186342A, which can detect 18 kinds of pathogens; CN111074001A, which can detect 9 kinds of pathogens. However, these techniques still face the problems of poor detectable rate and accuracy due to the complexity of the pathogens to be detected.

Therefore, there is a need in the art for a detection kit for rapidly and accurately detecting the most common respiratory tract infection pathogens with a high detectable rate, accuracy and sensitivity.

### SUMMARY

Accordingly, the detection of various viruses is required to be specifically configured in order to improve the detectable rate and accuracy. Taking the nucleic acid detection for Rhinovirus as an example, since both human Rhinovirus and enterovirus belong to the genus Enterovirus, many reagents or methods for the detection cannot distinguish between human Rhinovirus and enterovirus, or can distinguish them but with poor specificity. The reason why these reagents cannot accurately detect Rhinovirus is that only the conservative property of 5′ terminal untranslated region of the genus Enterovirus is considered when designing primers and probes, so Rhinovirus cannot be well differentiated from enterovirus. Against gene sequences of different mutation types of Rhinovirus, the inventors have designed two probes that can cover various mutation types of Rhinovirus without non-specific binding to enterovirus. By using such two probes with completely different sequences but the same fluorescent labels, the Rhinovirus can be detected accurately and effectively.

Taking the detection for Respiratory adenovirus as an example, two forward primers and two identical fluorescence-labeled probes are designed for various genotypes of adenovirus in order to ensure the accuracy of the detection results, since there are 52 serotypes of adenovirus among which types 3, 7, 11, 14, 16, 21, 34, 35, 50, 55 and so on capable of causing respiratory infections in humans, and recombination of 2 or more adenovirus strains may produce a new type of adenovirus.
In a first aspect, the disclosure provides a composition for detecting and identifying a pathogen that causes a respiratory tract infection, the composition including:
a first nucleic acid composition, including:
an Influenza A virus forward primer as set forth in SEQ ID NO:1, an Influenza A virus reverse primer as set forth in SEQ ID NO:2, and an Influenza A virus probe as set forth in SEQ ID NO:3;
an Influenza B virus forward primer as set forth in SEQ ID NO:4, an Influenza B virus reverse primer as set forth in SEQ ID NO:5, and an Influenza B virus probe as set forth in SEQ ID NO:6;
a Rhinovirus forward primer as set forth in SEQ ID NO:7, a Rhinovirus reverse primer as set forth in SEQ ID NO:8, a Rhinovirus probe as set forth in SEQ ID NO:9, and a Rhinovirus probe as set forth in SEQ ID NO:10; and
a second nucleic acid composition, including:
a Respiratory adenovirus forward primer as set forth in SEQ ID NO: 11, a Respiratory adenovirus forward primer as set forth in SEQ ID NO: 12, a Respiratory adenovirus reverse primer as set forth in SEQ ID NO: 13, a Respiratory adenovirus probe as set forth in SEQ ID NO: 14, and a Respiratory adenovirus probe as set forth in SEQ ID NO: 15;
a Respiratory syncytial virus forward primer as set forth in SEQ ID NO:16, a Respiratory syncytial virus reverse primer as set forth in SEQ ID NO:17, and a Respiratory syncytial virus probe as set forth in SEQ ID NO:18;
a Mycoplasma pneumoniae forward primer as set forth in SEQ ID NO:19, a Mycoplasma pneumoniae reverse primer as set forth in SEQ ID NO:20, and a Mycoplasma pneumoniae probe as set forth in SEQ ID NO:21;
wherein, fluorescent groups in the first group (the first nucleic acid composition) are different from each other and do not interfere with each other, and fluorescent groups in the second group (the second nucleic acid composition) are different from each other and do not interfere with each other.

The expression “different from each other and do not interfere with each other” herein means that the fluorescent groups used by respective probe in the composition are different, and will not affect the detection of each other, that is, can be detected using different. For example, FAM, HEX, ROX and CY5 may be used. These groups that do not have approximate absorbance values allow the selection of different channels, and thus will not interfere with each other.

In a further embodiment, the composition includes an internal standard forward primer, an internal standard reverse primer and an internal standard probe, that are configured for monitoring.

In one specific embodiment, the composition further includes an internal standard forward primer as set forth in SEQ ID NO: 22, an internal standard reverse primer as set forth in SEQ ID NO: 23, and an internal standard probe as set forth in SEQ ID NO: 24.

In the disclosure, the fluorescent reporter group may be selected from a group consisting of FAM, HEX, ROX, VIC, CY5, 5-TAMRA, TET, CY3 and JOE, but is not limited thereto.

In one specific embodiment, the fluorescent reporter group of the Influenza A virus probe as set forth in SEQ ID NO:3 is FAM; the fluorescent reporter group of the Influenza B virus probe as set forth in SEQ ID NO:6 is HEX; the fluorescent reporter group of the human Rhinovirus probe as set forth in SEQ ID NOs: 9 and 10 is CY5.

In one specific embodiment, the fluorescent reporter group of the Respiratory adenovirus probe as set forth in SEQ ID Nos: 14 and 15 is FAM; the fluorescent reporter group of the Respiratory syncytial virus probe as set forth in SEQ ID NO: 18 is HEX; the fluorescent reporter group of the Mycoplasma pneumoniae probe as set forth in SEQ ID NO: 21 is CY5.

In one specific embodiment, the fluorescent reporter group of the internal standard probe as set forth in SEQ ID NO: 24 is ROX.

The internal standard is the primers and probe that detect human housekeeping gene GAPDH gene sequence. GAPDH gene sequence (SEQ ID NO: 25) is: GGGGAGCCAAAAGGGTCATCATCTCTGCCCCCTCTGCTGATGCCCCCATGTTCGTCAT GGGTGTGAACCATGAGAAGTATGACAACAGCC.

In a further embodiment, the detection primers are used in the composition in an amount of 0.2-0.4 pM; the detection probes are used in the composition in an amount of 0.1-0.3 pM; the internal standard primers are used in the composition in an amount of 0.2 pM; and the internal standard probe is used in the composition in an amount of 0.1 pM.

In the disclosure, the terms “detection primers” and “detection probes” refer to the primers and probes for amplification and detection of a pathogen.

In the disclosure, the terms “internal standard primer” and “internal standard probe” refer to a primer and a probe for amplification and detection of an internal standard.

In one specific embodiment, two nucleic acid compositions of the composition of the disclosure are respectively present in separate packages.

In a further specific embodiment, each component in each nucleic acid composition of the compositions of the disclosure is present in a mixed form.

In a second aspect, the disclosure provides use of the above-mentioned composition of the disclosure in the preparation of a kit for detecting and identifying a pathogen that causes a respiratory tract infection.

The pathogen that causes a respiratory tract infection includes Influenza A virus, Influenza B virus, Respiratory adenovirus, human Rhinovirus, Respiratory syncytial virus and Mycoplasma pneumoniae.

In a third aspect, the disclosure provides a kit for detecting and identifying a pathogen that causes a respiratory tract infection, the kit including the above-mentioned composition of the disclosure.

In a further embodiment, the kit further includes a nucleic acid release reagent and a PCR amplification system.

In a further embodiment, the kit also includes at least one of a nucleic acid release reagent, dNTP, reverse transcriptase, DNA polymerase, a PCR buffer or Mg²⁺.

Common PCR buffers include Tris-HCl, MgCl2, KCl, Triton X-100 and other buffer systems. Generally, a total volume in a single PCR reaction tube is 20-2004

In a further embodiment, the detection primers are used in the composition in an amount of 25-500 nM; the detection probes are used in the composition in an amount of 20-500 nM; the internal standard primers are used in the composition in an amount of 12.5-250 nM; the internal standard probe is used in the composition in an amount of 10-250 nM; and dNTP are used is in an amount of 0.2-0.3 mM.

In further embodiment, the reverse transcriptase is at a concentration of 5 U/μL to 15 U/μL, and the reverse transcriptase may be, for example, murine leukemia reverse transcriptase (MMLV) or Tth enzyme. The DNA polymerase is at a concentration of 5 U/μL to 15 U /μL, and the DNA polymerase may be, for example, Taq enzyme.

In a further embodiment, the kit contains a positive control and a negative control. The negative and positive controls need to be processed simultaneously with the sample to be tested. The positive control, which simulates actual clinical specimens, is a mixture of artificially synthesized lentiviral particles containing specific nucleic acid sequences of Influenza A virus, Influenza B virus, human Rhinovirus and Respiratory syncytial virus, and artificially synthesized plasmids containing specific nucleic acid sequences of adenovirus and Mycoplasma pneumoniae. The negative control, which completely simulates throat swab samples of normal people, is composed of artificially synthesized lentiviral particles containing the target fragment (SEQ ID NO: 25) of GAPDH housekeeping gene.

In a fourth aspect, a method for detecting and identifying a pathogen that causes a respiratory tract infection is provided. The method includes the following steps:
1) releasing a nucleic acid in a sample to be detected;
2) performing a fluorescence quantitative PCR on the nucleic acid obtained in step 1) using the above-mentioned composition or the above-mentioned kit of the disclosure; and
3) obtaining a result and analyzing the result.

In the disclosure, the sample to be tested may be a throat swab, sputum, bronchoalveolar lavage fluid, blood and so on, but is not limited thereto.

In a further embodiment, the fluorescent quantitative PCR is performed under the following conditions:

1 cycle of pre-denaturation and enzyme activation at a temperature of 95° C. for 1 to 10 minutes; 1 cycle of reverse transcription at a temperature of 60° C. for 25 to 35 minutes; 1 cycle of cDNA pre-denaturation at a temperature of 95° C. for 1 to 10 minute; 40 to 50 cycles of denaturation at a temperature of 95° C. for 10 to 20 seconds and annealing at a temperature of 60° C. for 20 to 40 seconds.

In one specific embodiment, a method for detecting and identifying a pathogen that causes a respiratory tract infection for non-diagnostic purposes is provided. The method includes the following steps:
1) releasing a nucleic acid in a sample to be detected;
2) performing a fluorescence quantitative PCR on the nucleic acid obtained in step 1) using the above-mentioned composition or the above-mentioned kit of the disclosure; and
3) obtaining a result and analyzing the result.

The compositions of the disclosure can be used to simultaneously detect six pathogens that cause respiratory tract infection, enabling simple, quick, and objective detection of the respiratory tract pathogens, thereby realizing identification of pathogens, early diagnosis, rational guidance of clinical medication, reducing waste of medical resources, and reducing psychological burden on patients and society. At the same time, it has a higher detectable rate than that of the existing respiratory pathogen detection composition, and is more accurate in detection.

The composition of the disclosure, combined with the fluorescent probe method, enables the simultaneous use of two tubes in one test, and thus has low cost and high throughput. The information of four target points can be given in one tube in a single test with simple and convenient operation, and the results can be read by judgment with CT values. The whole process of the detection is carried out in a single tube under sealing, which avoids false positives and environmental contamination caused by cross-over between samples.

### BRIEF DESCRIPTION OF THE DRAWINGS

FIG. 1 is a graph showing the result of the detection using the composition of the disclosure.

FIG. 2 is a graph showing the result of the sensitivity detection of the compositions of the disclosure to Influenza A virus.

FIG. 3 is a graph showing the result of the sensitivity detection of the compositions of the disclosure to Influenza B virus.

FIG. 4 is a graph showing the result of the sensitivity detection of the compositions of the disclosure to human Rhinovirus.

FIG. 5 is a graph showing the result of the sensitivity detection of the compositions of the disclosure to Respiratory syncytial virus.

FIG. 6 is a graph showing the result of the sensitivity detection of the compositions of the disclosure to adenovirus.

FIG. 7 is a graph showing the result of the sensitivity detection of the compositions of the disclosure to Mycoplasma pneumoniae.

FIG. 8 is a graphs showing the specificity of the compositions of the disclosure.

FIG. 9 is a graphs showing the specificity of the compositions of the disclosure.

FIG. 10A is a graph showing the effect of the compositions of the disclosure.

FIG. 10B is a graph showing the effect when the Respiratory syncytial virus in the disclosure is replaced by SARS-CoV-2.

FIG. 11A is a graph showing the effect of the compositions of the disclosure.

FIG. 11B is a graph showing the effect when the Rhinovirus of the disclosure is replaced by metapneumovirus.

### DETAILED DESCRIPTION OF THE EMBODIMENTS

Hereinafter, the disclosure will be described in detail with reference to specific embodiments and examples, and the advantages and various effects of the disclosure will be more clearly presented therefrom. It is understood by those skilled in the art that these specific embodiments and examples are intended to illustrate, but not to limit the disclosure.

### EXAMPLE 1. PRIMERS AND PROBES USED IN THE DISCLOSURE

The primers and probes used in the disclosure are shown in Table 1 below (SEQ ID NOs: 1 to 24 corresponds to the base sequences only, and the labeling modes here are shown as specific examples only):

TABLE 1

| | Detection | |
|---|---|---|
| Name | target | Sequences and labeling modes (5′-3′) |
| SEQ ID NO: 1 | Influenza A | TCTCATGGAATGGCTAAAGACAA |
| SEQ ID NO: 2 | virus | AGGGCATTTTGGACAAAGC |
| SEQ ID NO: 3 | | FAM-CACCGTGCCCAGTGAGCGAGGA-BHQ1 |
| SEQ ID NO: 4 | Influenza B | TCACTCTTCGAGCGTTTTAATGAAG |
| SEQ ID NO: 5 | virus | TAATCGGTGCTCTTGACCAAAT |
| SEQ ID NO: 6 | | HEX-AGCCAATTCGAGCAGCTGAAACTGCG-BHQ1 |
| SEQ ID NO: 7 | Human | GCCCCTGAATGCGGCTAA |
| SEQ ID NO: 8 | Rhinovirus | GAAACACGGACACCCAAAGTAGT |
| SEQ ID NO: 9 | | CY5-TAATGAGCAATTGCGGGATGGGACC-BHQ2 |
| SEQ ID NO: 10 | | CY5-CTAGTGCATACAATCCAGTGTGTRGCTAG-BHQ2 |
| SEQ ID NO: 11 | Respiratory | GGATCCACCTCAAAAGTCAT |
| SEQ ID NO: 12 | adenovirus | TGGGCGGAGTTGGCGTAGA |
| SEQ ID NO: 13 | | GTTAAGAGTATTACCCAGAAAAAGTT |
| SEQ ID NO: 14 | | FAM-ACATGAAGTTGCTGGAGAAGGGGAT-BHQ1 |
| SEQ ID NO: 15 | | FAM-CCCATGGACATAAAGTTACTGGAGAAGG-BHQ1 |
| SEQ ID NO: 16 | Respiratory | AGAACAAGATGGGGCAAATATG |
| SEQ ID NO: 17 | syncytial virus | TATGTTGATGCTTGCAAGTTCTTTTAT |
| SEQ ID NO: 18 | | HEX-CGTGAACAAGCTTCACGAAGGCTCCACA-BHQ1 |
| SEQ ID NO: 19 | Mycoplasma | CCGTTTCTCCACCGGGTT |
| SEQ ID NO: 20 | pneumoniae | GTTTTAGGCGCGGTTATATCATC |
| SEQ ID NO: 21 | | CY5-CCCTGGATTGGGAATGGGTACAGGTATG-BHQ2 |
| SEQ ID NO: 22 | Human | GGGGAGCCAAAAGGGTCAT |
| SEQ ID NO: 23 | Housekeeping | GGCTGTTGTCATACTTCTCATGGTT |
| SEQ ID NO: 24 | Gene | ROX-ATCTCTGCCCCCTCTGCTGATGCCC-BHQ2 |
| | Internal | |
| | Standard | |

### EXAMPLE 2. METHODS FOR DETECTING PATHOGENS THAT CAUSE RESPIRATORY TRACT INFECTIONS AND IDENTIFYING THE PATHOGENS

In the disclosure, the samples to be detected were throat swabs, sputum and bronchoalveolar lavage fluid, and blood. Viral nucleic acid was extracted using the magnetic bead method. The following operation was performed in a sample processing chamber.
2.1. An appropriate number of 1.5 mL sterilized centrifuge tubes were labeled for negative control, positive control and samples to be tested, and 300 μL of RNA extraction solution 1 was added to each tube;
2.2. 200 μL of the sample to be tested, negative control or positive control was added per tube; and the tubes were capped, mixed by shaking for 10 seconds, and centrifuged instantaneously;
2.3. 100 μL of RNA extraction solution 2-mix (which should be mixed well before pipetted) was added to each tube, and the tubes were mixed by shaking for 10 seconds, then left standing at room temperature for 10 minutes;
2.4. After instantaneous centrifugation, the centrifuge tubes were placed on a separator for 3 minutes, and the solution was gently pipetted out (be careful not to touch the brown substance adsorbed on the tube wall);
2.5 600 μL of RNA extraction solution 3 and 200 μL of RNA extraction solution 4 were added to each tube, and the centrifuge tubes were mixed by shaking for 5 seconds, centrifuged instantaneously, and placed on the separator again;
2.6. After about 3 minutes, the supernatant was divided into two layers, a pipette tip was inserted into the bottom of the centrifuge tube to gently pipette the liquid out from the bottom completely and discard the liquid, and then the tube was left standing for 1 minute, and then the residual liquid at the bottom of the tube was pipetted out completely and discarded.
2.7. Elution was performed by adding 50μL of TE buffer (pH 8.0) to each tube, then all the brown mixture obtained after elution was transferred into a 0.2mL of PCR reaction tube as a sample to be tested, and then the tubes were capped, and transferred to an amplification detection area.

A real-time fluorescent PCR reaction system was configured as follows:

PCR reaction system A:

| | Volume/Concentration | |
|---|---|---|
| Components | in each reaction | |
| PCR buffer | 19.20 | μL |
| dNTP (100 mM) | 0.50 | μL |
| 1 mol/L MgCl₂ | 0.20 | μL |
| Primer SEQ ID NO: 1 (50 pmol/μL) | 0.40 | μL |
| Primer SEQ ID NO: 2 (50 pmol/μL) | 0.40 | μL |
| Primer SEQ ID NO: 4 (50 pmol/μL) | 0.40 | μL |
| Primer SEQ ID NO: 5 (50 pmol/μL) | 0.40 | μL |
| Primer SEQ ID NO: 7 (50 pmol/μL) | 0.30 | μL |
| Primer SEQ ID NO: 8 (50 pmol/μL) | 0.30 | μL |
| Primer SEQ ID NO: 22 (50 pmol/μL) | 0.2 | μL |
| Primer SEQ ID NO: 23 (50 pmol/μL) | 0.2 | μL |
| Probe SEQ ID NO: 3 (50 pmol/μL) | 0.20 | μL |
| Probe SEQ ID NO: 6 (50 pmol/μL) | 0.30 | μL |
| Probe SEQ ID NO: 9 (50 pmol/μL) | 0.20 | μL |
| Probe SEQ ID NO: 10 (50 pmol/μL) | 0.10 | μL |
| Probe SEQ ID NO: 24 (50 pmol/μL) | 0.10 | μL |
| RNasin | 0.125 | μL |

| | |
|---|---|
| 0.1% DEPC water | Make up to 43.5 μL |

PCR reaction system B:

| | Volume/Concentration | |
|---|---|---|
| Component | in each reaction | |
| PCR buffer | 19.20 | μL |
| dNTP(100 mM) | 0.5 | μL |
| 1 mol/L MgCl2 | 0.20 | μL |
| Primer SEQ ID NO: 11 (50 pmol/μL) | 0.40 | μL |
| Primer SEQ ID NO: 12 (50 pmol/μL) | 0.20 | μL |
| Primer SEQ ID NO: 13 (50 pmol/μL) | 0.40 | μL |
| Primer SEQ ID NO: 16 (50 pmol/μL) | 0.30 | μL |
| Primer SEQ ID NO: 17 (50 pmol/μL) | 0.30 | μL |
| Primer SEQ ID NO: 19 (50 pmol/μL) | 0.30 | μL |
| Primer SEQ ID NO: 20 (50 pmol/μL) | 0.30 | μL |
| Primer SEQ ID NO: 22 (50 pmol/μL) | 0.20 | μL |
| Primer SEQ ID NO: 23 (50 pmol/μL) | 0.20 | μL |
| Probe SEQ ID NO: 14 (50 pmol/μL) | 0.20 | μL |
| Probe SEQ ID NO: 15 (50 pmol/μL) | 0.10 | μL |
| Probe SEQ ID NO: 18 (50 pmol/μL) | 0.20 | μL |
| Probe SEQ ID NO: 21 (50 pmol/μL) | 0.20 | μL |
| Probe SEQ ID NO: 24 (50 pmol/μL) | 0.10 | μL |
| RNasin | 0.125 | μL |

| | |
|---|---|
| Sterilized purified water | Make up to 43.5 μL |

An enzyme mix consisting of Neoscript RT reverse transcriptase and H-Taq enzyme was obtained by mixing H-Taq enzyme (15 U/μL) and Neoscript RT enzyme (18 U/μL) according to a certain ratio (1 μL of H-Taq enzyme and 0.5 μL of Neoscript RT enzyme mix per person).

A PCR amplification program was set up as follows:

| | | | | | Cycle |
|---|---|---|---|---|---|
| Step | | Temperature | Time | | Number |
| 1 | reverse transcription | 50° C. | 30 | minutes | 1 |
| 2 | pre-denaturation and | 95° C. | 1 | minute | 1 |
| | enzyme activation | | | | |
| 3 | denaturation | 95° C. | 15 | seconds | 45 |
| | annealing, extension and | 60° C. | 30 | seconds | |
| | fluorescence acquisition | | | | |
| 4 | instrument cooling | 25° C. | 10 | seconds | 1 |

Result analysis:
1) the detection signal for the target was FAM, HEX (or VIC) and CYS, and the detection signal for internal reference was ROX;
2) Baseline setting: Baseline was generally set to 3-15 cycles, which could be adjusted according to the actual situation. The adjustment principle was to select a region where fluorescence signal is relatively stable before the exponential amplification part, with a start point (Start) avoiding the signal fluctuation in the initial stage of fluorescence acquisition, and an end point (End) being less by 1-2 cycles than the Ct of a sample showing the earliest exponential amplification. Threshold setting: the setting principle was to make a threshold line just exceeded the highest point of a normal negative control.

A sample was confirmed positive if obvious sigmoid amplification curves occurred in FAM, HEX and CY5 channels and the Ct value was less than or equal to 40; and a sample was confirmed negative if no amplification curve occurs in FAM, HEX and CY5 channels (that is, No Ct) or the Ct value was greater than 40, and the ROX internal standard channel was positive (Ct value ≤40). The details were as follows:

| Amplification results | FAM channel | | HEX channel | |
|---|---|---|---|---|
| reaction solution | Ct value ≤ 40 | No Ct or Ct | Ct value ≤ 40 | No Ct or Ct |
| | | value > 40 | | value > 40 |
| PCR mix A | Flu A positive | Flu A negative | Flu B positive | Flu B negative |
| PCR mix B | ADV positive | ADV negative | RSV positive | RSV negative |
| Amplification results | CY5 channel | | ROX channel | |
| reaction solution | Ct value ≤ 40 | No Ct or Ct | Ct value ≤ 40 | No Ct or Ct |
| | | value > 40 | | value > 40 |
| PCR mix A | HRV positive | HRV negative | Internal | Internal |
| | | | Standard | Standard |
| | | | positive | negative |
| PCR mix B | MP positive | MP negative | | |

### EXAMPLE 3. DETECTABLE RATE OF THE COMPOSITIONS OF THE DISCLOSURE

The detection was carried out using the compositions in Table 1 according to the method described in Example 2 of the disclosure, and a series of controls (other dual-probe schemes: probe 1 (SEQ ID NO:26): CY5-CTAACCTTAACCCCGCAGC-BHQ2; probe 2 (SEQ ID NO:27): CY5-AATCCTAACCATGGAGCAAG- BHQ) were also designed for the detection as well. The detection results are shown in Tables 2 and 3.

TABLE 2
Statistical table of Ct value results of
different Rhinovirus detection schemes

| | Scheme | | Single- | Sequencing results |
|---|---|---|---|---|
| Sample | of the | Other dual- | probe | of 5′ untranslated |
| number | disclosure | probe schemes | scheme | region |
| 1 | 13.88 | 26.13 | 17.76 | Rhinovirus A |
| 2 | 22.81 | 34.03 | 32.22 | Rhinovirus A39 |
| 3 | 31.40 | 40.47 | No Ct | Rhinovirus A |
| 4 | No Ct | 31.82 | 23.9 | Coxsackievirus EV71 |
| 5 | 29.03 | 33.38 | No Ct | Rhinovirus C |
| 6 | 22.33 | 31.62 | 24.79 | Rhinovirus A22 |
| 7 | 39.89 | No Ct | No Ct | Rhinovirus A22 |
| 8 | 19.11 | 28.55 | 21.21 | Rhinovirus A22 |
| 9 | 21.66 | 32.13 | No Ct | Rhinovirus A |
| 10 | 19.18 | 24.28 | 21.8 | Rhinovirus A |
| 11 | 39.67 | No Ct | No Ct | Rhinovirus A |
| 12 | 39.68 | 37.64 | No Ct | Rhinovirus A |
| 13 | 31.46 | 38.04 | No Ct | Rhinovirus A10 |
| 14 | 26.90 | No Ct | No Ct | Rhinovirus A |
| 15 | 24.28 | 32.12 | 28.19 | Rhinovirus A46 |
| 16 | 19.66 | 30.85 | 23.33 | Rhinovirus A103 |
| 17 | 23.78 | 27.22 | 25.31 | Rhinovirus A |
| 18 | 25.71 | 36.52 | No Ct | Rhinovirus A |
| 19 | 24.18 | 30.43 | 26.32 | Rhinovirus A22 |
| 20 | No Ct | No Ct | 30.79 | Coxsackievirus A6 |

Notes:
a larger Ct value means a worse detection effect, and No Ct means negative.

It could be seen from the detection results in the above table that, enteroviruses (Coxsackievirus EV71, Coxsackievirus A6) would not be detected by using the scheme of the disclosure; while for Rhinovirus positive samples, Ct values obtained by detecting with the scheme of the disclosure were smaller, indicating that it has better detection effect than that of the other dual-probe scheme and single-probe scheme.

TABLE 3
Summary of Ct value results of different adenovirus detection schemes

| | | | | The disclosure |
|---|---|---|---|---|
| | The | The disclosure | The disclosure | scheme without |
| Sample | disclosure | scheme without | scheme without | SEQ ID NO: 12 |
| Number | Scheme | SEQ ID NO: 12 | SEQ ID NO: 15 | and SEQ ID NO: 15 |
| 1 | 31.08 | 29.17 | 41.27 | No Ct |
| 2 | 33.16 | No Ct | No Ct | No Ct |
| 3 | 34.92 | 33.89 | No Ct | No Ct |
| 4 | 34.56 | 34.84 | No Ct | No Ct |
| 5 | 25.89 | 37.27 | No Ct- | No Ct |
| 6 | 34.36 | No Ct | No Ct | No Ct |
| 7 | 21.55 | 21.72 | 21.48 | 32.72 |
| 8 | 20.37 | 20.45 | 20.78 | 40.89 |
| 9 | 34.29 | 33.79 | No Ct | No Ct |
| 10 | 36.09 | 36.24 | 33.16 | No Ct |
| 11 | 20.51 | 20.63 | 20.97 | No Ct |
| 12 | 23.74 | 23.70 | 23.94 | 34.04 |
| 13 | 22.45 | 22.63 | 22.95 | No Ct |
| 14 | 35.43 | 35.24 | 35.25 | No Ct |
| 15 | 26.84 | 26.70 | 27.83 | 37.85 |
| 16 | 34.78 | 34.24 | 34.65 | No Ct |
| 17 | 34.20 | 33.59 | 33.25 | 38.15 |
| 18 | 23.01 | 22.33 | 23.25 | 26.09 |
| 19 | 31.62 | 31.47 | 32.08 | 32.38 |
| 20 | 30.65 | 29.76 | 31.17 | 31.84 |
| 21 | 28.43 | 29.61 | 28.11 | 29.42 |
| 22 | 30.29 | 30.82 | 30.26 | 33.56 |

Note:
a larger Ct value means a worse detection effect, and No Ct means negative.

It could be seen from the detection results in the above table that, all 22 positive samples could be detected by using the compositions of the disclosure; where there was no primer SEQ ID NO: 12, 2 positive samples were not detected; where there was no probe SEQ ID NO: 15, 6 positive samples were not detected; where there was neither SEQ ID NO: 12 nor SEQ ID NO: 15, 13 positive samples were not detected.

### EXAMPLE 4. SENSITIVITY OF THE COMPOSITIONS OF THE DISCLOSURE

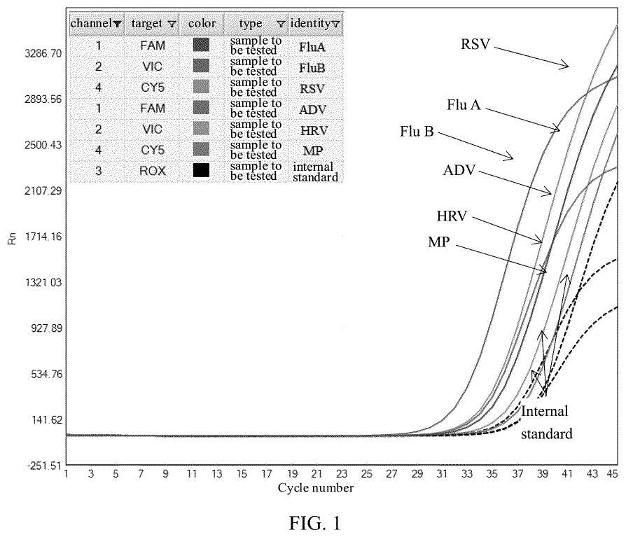

Six pathogens were tested using the compositions of the disclosure, and the detection results are shown in FIG. 1, indicating that the compositions of the disclosure were capable of detecting six pathogens.

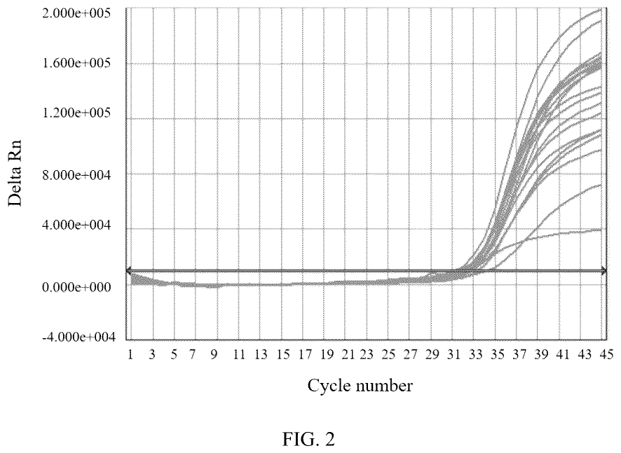

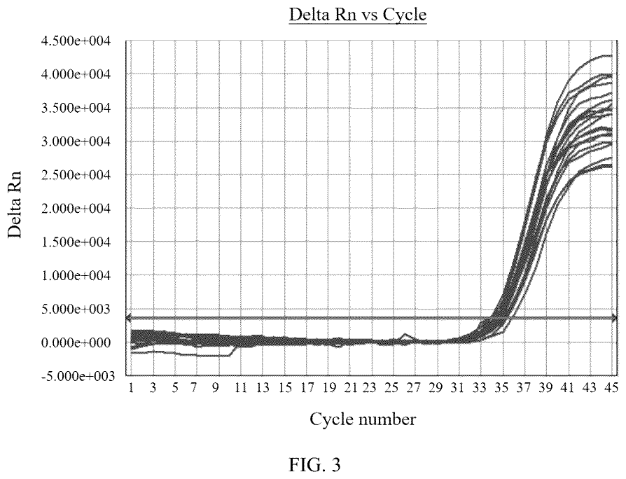

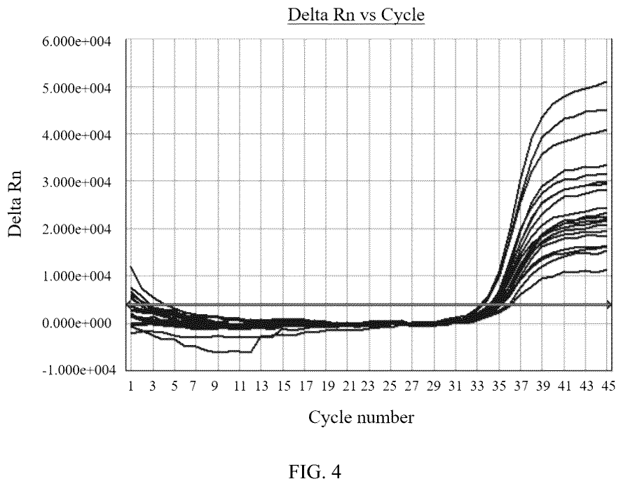

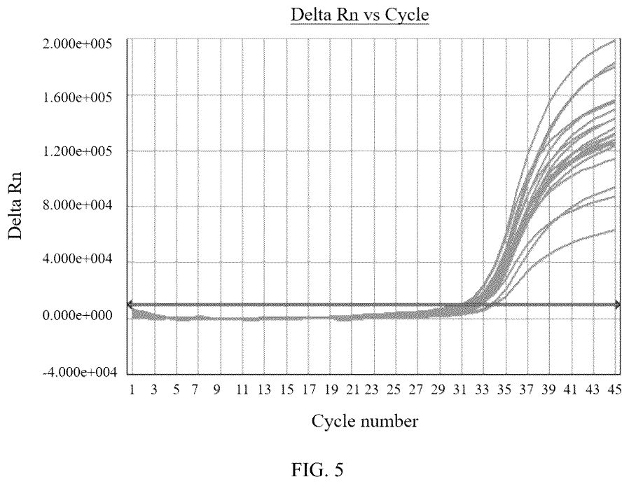

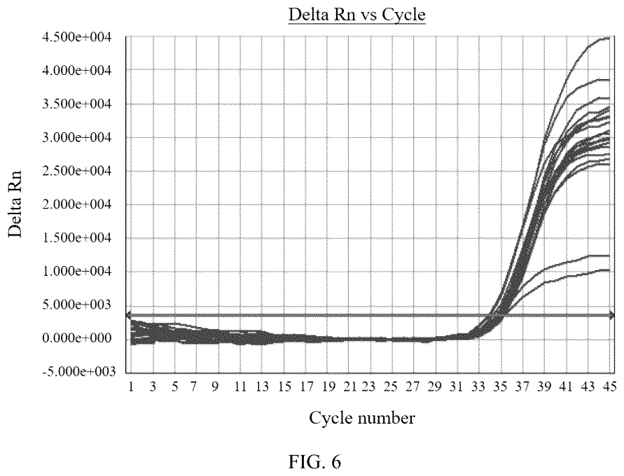

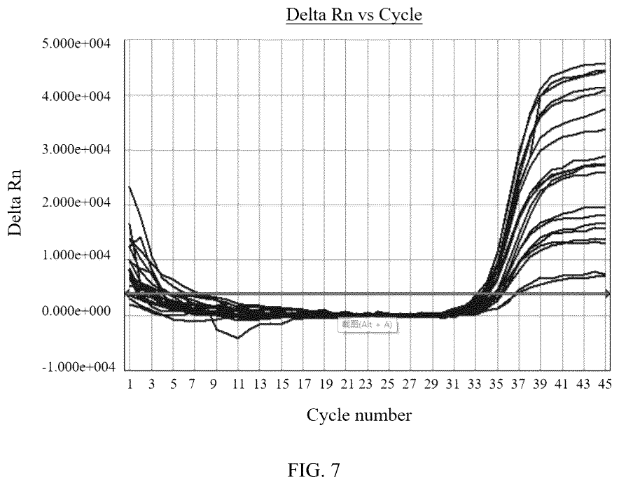

In addition, experiments were carried out with samples at different concentrations to detect the compositions of the disclosure for sensitivity using the compositions in Table 1 according to the method described in Example 2 of the disclosure. The positive samples for each target sensitivity were diluted in detection gradient, and the samples diluted to the detection limit were used as the test samples for detection, and each channel was detected 20 times. The analysis of sensitivity test results showed that, detection limits of this kit for Influenza A virus, Influenza B virus, human Rhinovirus, Respiratory syncytial virus, adenovirus and Mycoplasma pneumoniae were respectively: 2.0 TCID50/mL, 2.0 TCID50/mL, 500.0 copies/mL, 500.0 copies/mL, 500.0 copies/mL, 500.0 copies/mL, as shown in FIGS. 2-7.

### EXAMPLE 5. SPECIFICITY OF THE COMPOSITIONS OF THE DISCLOSURE

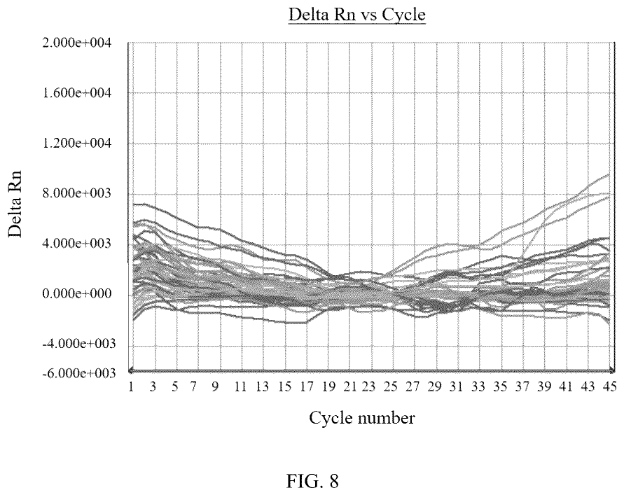

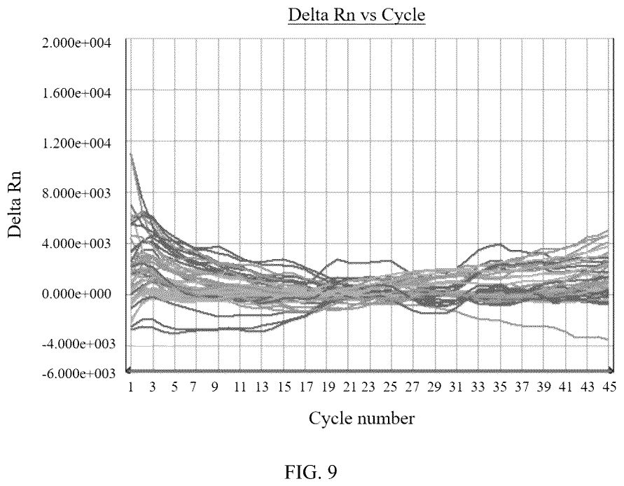

The compositions of the disclosure invention were used to detect other common respiratory pathogens. The experiment showed that the compositions of the disclosure had no cross-reactivity with common respiratory pathogens (measles virus, mumps virus, rubella virus, Staphylococcus aureus, Escherichia coli, Pseudomonas aeruginosa, human parainfluenza virus I, metapneumovirus, enterovirus 71, coronavirus 229E, cytomegalovirus, Coxsackievirus A, Bordetella pertussis, chlamydia pneumoniae, Haemophilus influenzae, Streptococcus salivarius, Streptococcus pneumoniae, Neisseria meningitidis, Mycobacterium tuberculosis, human parainfluenza viruses 2 and 3, Epstein-Barr virus, Cryptococcus, Aspergillus fumigatus, Aspergillus flavus, Candida albicans, Legionella pneumophila, Boca Virus, herpes simplex virus 1, varicella zoster virus, Corynebacterium diphtheria, Lactobacillus bulgaricus, Moraxella catarrhalis, Staphylococcus epidermidis, Streptococcus pyogenes, Pneumocystis, Klebsiella pneumoniae and so on). The experimental results are shown in FIGS. 8-9 (FIG. 8 shows the result of PCR reaction system A, and FIG. 9 shows the result of PCR reaction system B).

### EXAMPLE 6. JOINT DETECTION OF MULTIPLE PATHOGENS, MUTUAL INTERFERENCE BETWEEN PRIMERS AND PROBES

Due to the principle of complementary base pairing, dimers would form between primers and/or probes, but this probability was rare and could be ruled out at the beginning of the design. However, when multiple pathogens were jointly detected, there were many primers and probes, and dimers were prone to form between primers and primers, between probes and probes, or between primers and probes. To ensure the conservative property of the design (the conservative property was crucial to the accuracy of detection), and to consider the mutual interference between various primers and probes, the primers and probes needed to be carefully designed.

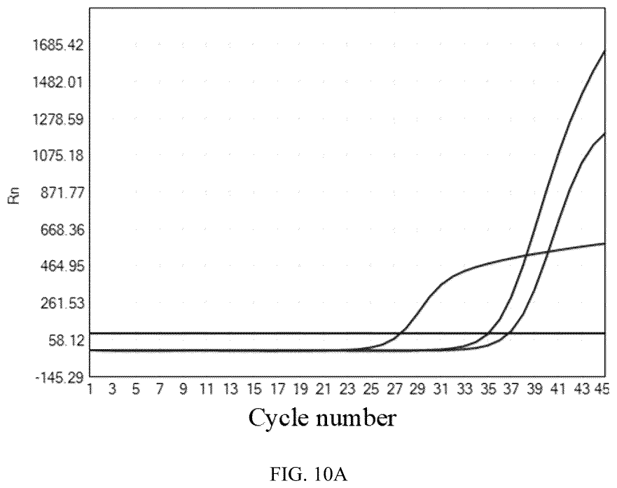

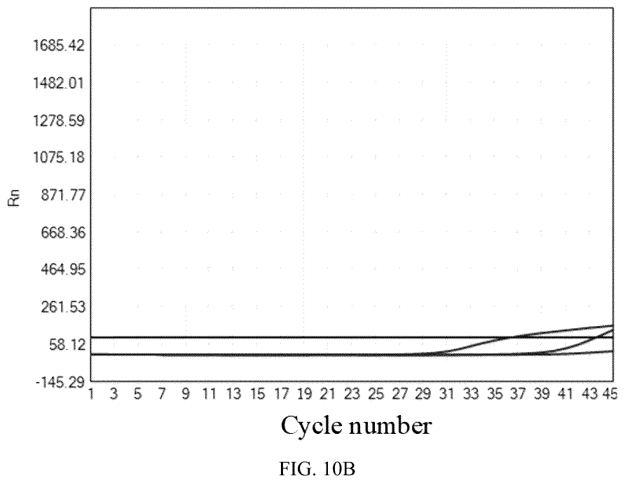

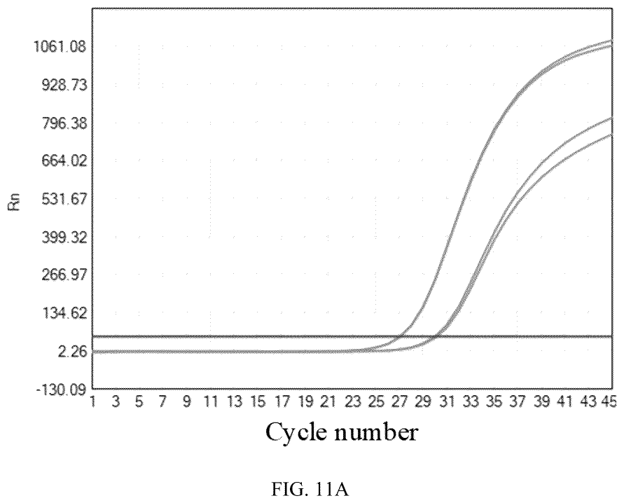

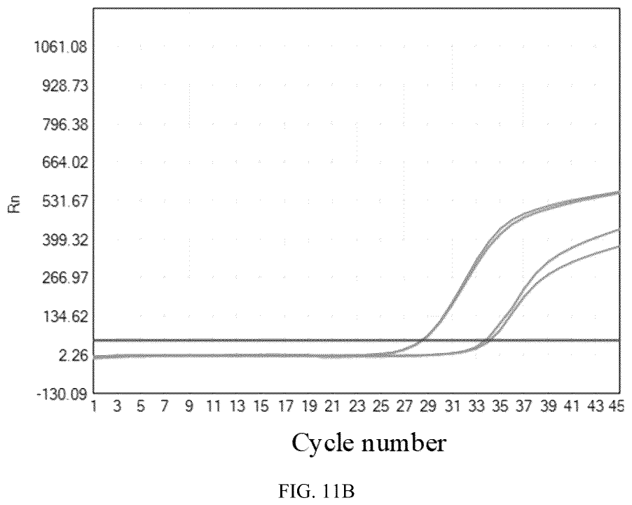

In the disclosure, the inventors replaced the Respiratory syncytial virus in the disclosure with SARS-CoV-2, or replaced the Rhinovirus in the disclosure with metapneumovirus. However, upon the experiments, it was found that the addition of SARS-CoV-2 and metapneumovirus would seriously affect the detection sensitivity of the method of the disclosure (experimental results are shown in FIGS. 10-11).

What is claimed is:

1. A composition for detecting and identifying a pathogen that causes a respiratory tract infection, comprising:
a first nucleic acid composition comprising
an Influenza A virus forward primer as set forth in SEQ ID NO:1, an Influenza A virus reverse primer as set forth in SEQ ID NO:2, and an influenza A probe as set forth in SEQ ID NO:3;
an Influenza B virus forward primer as set forth in SEQ ID NO:4, an Influenza B virus reverse primer as set forth in SEQ ID NO:5, and an influenza B probe as set forth in SEQ ID NO:6;
a Rhinovirus forward primer as set forth in SEQ ID NO:7, a Rhinovirus reverse primer as set forth in SEQ ID NO:8, a Rhinovirus probe as set forth in SEQ ID NO:9, and a Rhinovirus probe as set forth in SEQ ID NO:10; and
a second nucleic acid composition comprising
a Respiratory adenovirus forward primer as set forth in SEQ ID NO: 11, a Respiratory adenovirus forward primer as set forth in SEQ ID NO: 12, a Respiratory adenovirus reverse primer as set forth in SEQ ID NO: 13, a Respiratory adenovirus probe as set forth in SEQ ID NO: 14, and a Respiratory adenovirus probe as set forth in SEQ ID NO: 15;
a Respiratory syncytial virus forward primer as set forth in SEQ ID NO:16, a Respiratory syncytial virus reverse primer as set forth in SEQ ID NO:17, and a Respiratory syncytial virus probe as set forth in SEQ ID NO:18; and
a Mycoplasma pneumoniae forward primer as set forth in SEQ ID NO:19, a Mycoplasma pneumoniae reverse primer as set forth in SEQ ID NO:20, and a Mycoplasma pneumoniae probe as set forth in SEQ ID NO:21;
wherein, fluorescent groups in the first nucleic acid composition are different from each other and do not interfere with each other, and fluorescent groups in the second nucleic acid composition are different from each other and do not interfere with each other.

2. The composition of claim 1, wherein the composition further comprises an internal standard forward primer, an internal standard reverse primer and an internal standard probe, that are configured for monitoring.

3. The composition of claim 1, wherein the composition further comprises an internal standard forward primer as set forth in SEQ ID NO: 22, an internal standard reverse primer as set forth in SEQ ID NO: 23, and an internal standard probe as set forth in SEQ ID NO: 24.

4. The composition of claim 1, wherein the fluorescent group may be selected from a group consisting of FAM, HEX, ROX, VIC, CY5, 5-TAMRA, TET, CY3 and JOE.

5. The composition of claim 1, wherein a fluorescent reporter group of the Influenza A virus probe as set forth in SEQ ID NO:3 is FAM; and/or a fluorescent reporter group of the Influenza B virus probe as set forth in SEQ ID NO:6 is HEX; and/or a fluorescent reporter group of the human Rhinovirus probe as set forth in SEQ ID NOs: 9 and 10 is CY5.

6. The composition of claim 1, wherein a fluorescent reporter group of the Respiratory adenovirus probe as set forth in SEQ ID Nos: 14 and 15 is FAM; and/or a fluorescent reporter group of the Respiratory syncytial virus probe as set forth in SEQ ID NO: 18 is HEX; and/or a fluorescent reporter group of the Mycoplasma pneumoniae probe as set forth in SEQ ID NO: 21 is CY5.

7. The composition of claim 1, wherein two nucleic acid compositions of the composition are respectively present in separate packages.

8. The composition of claim 1, wherein the detection primers in the composition in an amount of 0.2-0.4 pM; and/or the detection probes are used in the composition in an amount of 0.1-0.3 pM.

9. A method for preparation a kit for detecting a pathogen that causes a respiratory tract infection by using the composition of claim 1.

10. The method of claim 1, wherein the pathogen that causes a respiratory tract infection comprises Influenza A virus, Influenza B virus, Respiratory adenovirus, human Rhinovirus, Respiratory syncytial virus and Mycoplasma pneumoniae.

11. A kit for detecting a pathogen that causes a respiratory tract infection and identifying the pathogen, comprising the composition of claim 1.

12. The kit of claim 11, wherein the kit further comprises a nucleic acid release reagent and a PCR amplification system.

13. A method for detecting and identifying a pathogen that causes a respiratory tract infection, comprising:
1) releasing a nucleic acid in a sample to be detected;
2) performing a fluorescence quantitative PCR on the nucleic acid obtained in step 1) using the composition of any one of claims 1; and
3) obtaining a result and analyzing the result.

14. A method for detecting and identifying a pathogen that causes a respiratory tract infection, comprising:
1) releasing a nucleic acid in a sample to be detected;
2) performing a fluorescence quantitative PCR on the nucleic acid obtained in step 1) using the kit of claim 11; and
3) obtaining a result and analyzing the result.

15. A composition for detecting and identifying a pathogen that causes a respiratory tract infection, comprising:
a first nucleic acid composition:
an Influenza A virus forward primer as set forth in SEQ ID NO:1, an Influenza A virus reverse primer as set forth in SEQ ID NO:2, and an influenza A probe as set forth in SEQ ID NO:3;
an Influenza B virus forward primer as set forth in SEQ ID NO:4, an Influenza B virus reverse primer as set forth in SEQ ID NO:5, and an influenza B probe as set forth in SEQ ID NO:6; and
a Rhinovirus forward primer as set forth in SEQ ID NO:7, a Rhinovirus reverse primer as set forth in SEQ ID NO:8, a Rhinovirus probe as set forth in SEQ ID NO:9, and a Rhinovirus probe as set forth in SEQ ID NO:10;
wherein, fluorescent groups in the first nucleic acid composition are different from each other and do not interfere with each other.

16. The composition of claim 15, wherein a fluorescent reporter group of the Influenza A virus probe as set forth in SEQ ID NO:3 is FAM; and/or a fluorescent reporter group of the Influenza B virus probe as set forth in SEQ ID NO:6 is HEX; and/or a fluorescent reporter group of the human Rhinovirus probe as set forth in SEQ ID NOs: 9 and 10 is CYS.

17. A kit for detecting a pathogen that causes a respiratory tract infection and identifying the pathogen, comprising the composition of claim 15.

18. A composition for detecting and identifying a pathogen that causes a respiratory tract infection, comprising:
a second nucleic acid composition:
a Respiratory adenovirus forward primer as set forth in SEQ ID NO: 11, a Respiratory adenovirus forward primer as set forth in SEQ ID NO: 12, a Respiratory adenovirus reverse primer as set forth in SEQ ID NO: 13, a Respiratory adenovirus probe as set forth in SEQ ID NO: 14, and a Respiratory adenovirus probe as set forth in SEQ ID NO: 15;
a Respiratory syncytial virus forward primer as set forth in SEQ ID NO:16, a Respiratory syncytial virus reverse primer as set forth in SEQ ID NO:17, and a Respiratory syncytial virus probe as set forth in SEQ ID NO:18; and
a Mycoplasma pneumoniae forward primer as set forth in SEQ ID NO:19, a Mycoplasma pneumoniae reverse primer as set forth in SEQ ID NO:20, and a Mycoplasma pneumoniae probe as set forth in SEQ ID NO:21;
wherein, fluorescent groups in the second nucleic acid composition are different from each other and do not interfere with each other.

19. The composition of claim 18, wherein a fluorescent reporter group of the Respiratory adenovirus probe as set forth in SEQ ID Nos: 14 and 15 is FAM; and/or a fluorescent reporter group of the Respiratory syncytial virus probe as set forth in SEQ ID NO: 18 is HEX; and/or a fluorescent reporter group of the Mycoplasma pneumoniae probe as set forth in SEQ ID NO: 21 is CY5.

20. A kit for detecting a pathogen that causes a respiratory tract infection and identifying the pathogen, comprising the composition of claim 18.


SEQUENCE LISTING (27 sequences)

SEQ ID NO: 1 - DNA, 23 bp, synthetic construct
  source 1..23: mol_type = other DNA; note = Influenza A forward primer
tctcatggaatggctaaagacaa

SEQ ID NO: 2 - DNA, 19 bp, synthetic construct
  source 1..19: mol_type = other DNA; note = Influenza A reverse primer
agggcattttggacaaagc

SEQ ID NO: 3 - DNA, 22 bp, synthetic construct
  source 1..22: mol_type = other DNA; note = Influenza A probe
caccgtgcccagtgagcgagga

SEQ ID NO: 4 - DNA, 25 bp, synthetic construct
  source 1..25: mol_type = other DNA; note = Influenza B forward primer
tcactcttcgagcgttttaatgaag

SEQ ID NO: 5 - DNA, 22 bp, synthetic construct
  source 1..22: mol_type = other DNA; note = Influenza B reverse primer
taatcggtgctcttgaccaaat

SEQ ID NO: 6 - DNA, 26 bp, synthetic construct
  source 1..26: mol_type = other DNA; note = Influenza B probe
agccaattcgagcagctgaaactgcg

SEQ ID NO: 7 - DNA, 18 bp, synthetic construct
  source 1..18: mol_type = other DNA; note = Rhinovirus forward primer
gcccctgaatgcggctaa

SEQ ID NO: 8 - DNA, 23 bp, synthetic construct
  source 1..23: mol_type = other DNA; note = Rhinovirus reverse primer
gaaacacggacacccaaagtagt

SEQ ID NO: 9 - DNA, 25 bp, synthetic construct
  source 1..25: mol_type = other DNA; note = Rhinovirus probe
taatgagcaattgcgggatgggacc

SEQ ID NO: 10 - DNA, 29 bp, synthetic construct
  source 1..29: mol_type = other DNA; note = Rhinovirus probe
ctagtgcatacaatccagtgtgtrgctag

SEQ ID NO: 11 - DNA, 20 bp, synthetic construct
  source 1..20: mol_type = other DNA; note = Respiratory adenovirus forward primer
ggatccacctcaaaagtcat

SEQ ID NO: 12 - DNA, 19 bp, synthetic construct
  source 1..19: mol_type = other DNA; note = Respiratory adenovirus forward primer
tgggcggagttggcgtaga

SEQ ID NO: 13 - DNA, 26 bp, synthetic construct
  source 1..26: mol_type = other DNA; note = Respiratory adenovirus forward primer
gttaagagtattacccagaaaaagtt

SEQ ID NO: 14 - DNA, 25 bp, synthetic construct
  source 1..25: mol_type = other DNA; note = Respiratory adenovirus probe
acatgaagttgctggagaaggggat

SEQ ID NO: 15 - DNA, 28 bp, synthetic construct
  source 1..28: mol_type = other DNA; note = Respiratory adenovirus probe
cccatggacataaagttactggagaagg

SEQ ID NO: 16 - DNA, 22 bp, synthetic construct
  source 1..22: mol_type = other DNA; note = Respiratory syncytial virus forward primer
agaacaagatggggcaaatatg

SEQ ID NO: 17 - DNA, 27 bp, synthetic construct
  source 1..27: mol_type = other DNA; note = Respiratory syncytial virus forward primer
tatgttgatgcttgcaagttcttttat

SEQ ID NO: 18 - DNA, 28 bp, synthetic construct
  source 1..28: mol_type = other DNA; note = Respiratory syncytial virus probe
cgtgaacaagcttcacgaaggctccaca

SEQ ID NO: 19 - DNA, 18 bp, synthetic construct
  source 1..18: mol_type = other DNA; note = mycoplasma pneumoniae forward primer
ccgtttctccaccgggtt

SEQ ID NO: 20 - DNA, 23 bp, synthetic construct
  source 1..23: mol_type = other DNA; note = mycoplasma pneumoniae reverse primer
gttttaggcgcggttatatcatc

SEQ ID NO: 21 - DNA, 28 bp, synthetic construct
  source 1..28: mol_type = other DNA; note = mycoplasma pneumoniae probe
ccctggattgggaatgggtacaggtatg

SEQ ID NO: 22 - DNA, 19 bp, synthetic construct
  source 1..19: mol_type = other DNA; note = internal standard forward primer
ggggagccaaaagggtcat

SEQ ID NO: 23 - DNA, 25 bp, synthetic construct
  source 1..25: mol_type = other DNA; note = internal standard reverse primer
ggctgttgtcatacttctcatggtt

SEQ ID NO: 24 - DNA, 25 bp, synthetic construct
  source 1..25: mol_type = other DNA; note = internal standard probe
atctctgccccctctgctgatgccc

SEQ ID NO: 25 - DNA, 90 bp, Homo sapiens
  source 1..90: mol_type = genomic DNA
ggggagccaaaagggtcatcatctctgccccctctgctgatgcccccatgttcgtcatgggtgtgaaccatgagaagtatgacaacagcc

SEQ ID NO: 26 - DNA, 19 bp, synthetic construct
  source 1..19: mol_type = other DNA; note = Human Rhinovirus probe 1
ctaaccttaaccccgcagc

SEQ ID NO: 27 - DNA, 20 bp, synthetic construct
  source 1..20: mol_type = other DNA; note = Human Rhinovirus probe 2
aatcctaaccatggagcaag